(19) United States
(12) Patent Application Publication
(10) Pub. No.: US 2017/0252388 A1
(43) Pub. Date: Sep. 7, 2017

(54) BEVERAGES FOR ENHANCING SEXUAL PERFORMANCE

(71) Applicant: HYDRO ONE LLC, Augusta, GA (US)

(72) Inventor: Babak C. BABAN, Augusta, GA (US)

(73) Assignee: HYDRO ONE LLC, Augusta, GA (US)

(21) Appl. No.: 15/450,103

(22) Filed: Mar. 6, 2017

Related U.S. Application Data
(60) Provisional application No. 62/304,394, filed on Mar. 7, 2016.

(51) Int. Cl. A61K 36/31; A61K 36/258; A23L 33/00; A61K 9/00; A23L 2/02; A23L 2/52; A61K 36/185; A61K 31/198

(52) U.S. Cl. CPC A61K 36/31; A61K 36/185; A61K 36/258; A61K 31/198; A61K 9/0095; A23L 2/02; A23L 2/52; A23L 33/30; A23V 2002/00

(57) ABSTRACT

Described herein are beverages that can enhance sexual performance in both men and women. For example, the beverages are useful in promoting endurance and stamina and in increasing sexual desire as well as blood flow to the genital region. Components of the beverages are also associated with a reduced risk of cardiovascular disease in subjects who consume the beverages. The beverages are composed of pomegranate juice, ginseng, maca root, and L-arginine or a pharmaceutically acceptable salt thereof. The beverages may additionally include B complex vitamins including folic acid, as well as Tongkat Ali and/or damiana leaf. Vitamins, minerals, other nutrients, and other natural products can also be added to the beverages to provide additional health benefits.

### CROSS REFERENCE TO RELATED APPLICATIONS

This application claims priority upon U.S. provisional application Ser. No. 62/304,394, filed Mar. 7, 2016. This application is hereby incorporated by reference in its entirety for all of its teachings.

### BACKGROUND

Sexual dysfunction in both men and women is characterized by recurring problems with sexual response or desire. Men may experience erectile dysfunction, premature ejaculation, or priapism, for example, and women may suffer from pain during intercourse due to insufficient lubrication, among other issues. In addition to sexual dysfunction, sexual dissatisfaction is common to both sexes. This dissatisfaction may not be classifiable as a medical dysfunction and thus may not be treatable by currently available pharmaceutical preparations. Sexual dissatisfaction and dysfunction are often associated with a decrease in the overall mental health or well-being of the individual and his or her partner. Thus, a non-prescription supplement for enhancing sexual performance, including overcoming some of the problems previously mentioned, would be advantageous from a variety of standpoints.

Many herbs and neutraceutical supplements for enhancing male sexual performance are available and similar preparations for women are becoming increasingly common. However, these supplements are generally packaged as pills or single-ingredient powders or teas. Subject compliance with consumption directions for pills is often low, and consuming only a single ingredient in powdered form or via a tea would not offer all the benefits of multiple ingredients. It would thus be advantageous to have available a multi-ingredient beverage designed to enhance sexual performance.

Further, cardiovascular disease is an increasing concern. Short term expense for hospitalization following a heart attack, for example, are extremely high and may include everything from medications to surgery. High blood pressure, atherosclerosis, angina, and other cardiovascular diseases are similarly on the rise and similarly require expensive medical interventions. Additional costs come from lost income due to time off work for cardiac patients. It would thus be advantageous, both economically and for the health of the individual consumer, if the beverage offered some reduction in risk for cardiovascular disease as well as address issues of sexual dysfunction discussed above.

Finally, it would be advantageous for the beverage to have a satisfactory taste so that the subject is encouraged to consume the beverage on a routine basis, for maximum reduction of risk of cardiovascular disease as well as to maintain the enhanced level of sexual performance imparted by consumption of the beverage. The present invention addresses these needs.

### SUMMARY

Described herein are beverages that can enhance sexual performance in both men and women. For example, the beverages are useful in promoting endurance and stamina and in increasing sexual desire as well as blood flow to the genital region. Components of the beverages are also associated with a reduced risk of cardiovascular disease in subjects who consume the beverages. The beverages are composed of pomegranate juice, ginseng, maca root, and L-arginine or a pharmaceutically acceptable salt thereof. The beverages may additionally include Tongkat Ali and/or damiana leaf. Vitamins, minerals, other nutrients, and other natural products can also be added to the beverages to provide additional health benefits.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “an antioxidant” includes mixtures of two or more such antioxidants, and the like.

“Optional” or “optionally” means that the subsequently described event or circumstance can or cannot occur, and that the description includes instances where the event or circumstances occurs and instances where it does not. For example, the phrase “optionally includes an extract of Tongkat Ali” means that the extract may or may not be present.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight component Y, X and Y are present at a weight ratio of 2:5, and are present in such ratio regardless of whether additional components are contained in the composition.

A weight percent of a component, unless specifically stated to the contrary, is based on the total weight of the formulation or composition in which the component is included.

The beverages described herein are composed of pomegranate juice, ginseng, maca, and L-arginine or a pharmaceutically acceptable salt thereof and optionally Tongkat Ali and/or damiana leaf. Each component of the beverages described herein and their preparation and use thereof is described below.

Pomegranate juice forms a base for the beverage and is a natural source of several compounds that impart numerous health benefits. In one aspect, the pomegranate juice is the juice that is pressed from ripe pomegranate. Thus, the juice is taken directly from the fruit and is not a concentrate. Moreover, no additives or sweeteners are added to the juice.

Pomegranate juice includes natural antioxidants. An “antioxidant” is a molecule that inhibits the oxidation of one or more other molecules. During the process of oxidation, free radicals can be produced, and these may damage cells. An antioxidant can terminate these types of reaction. Antioxidants include vitamins (such as, for example, vitamin A in retinol or beta-carotene form, vitamin C, and/or vitamin E); metabolites such as glutathione, coenzyme Q10, melatonin, or uric acid, or enzymes (e.g., catalase, superoxide dismutase, etc. The antioxidants present in pomegranate juice can also extend the shelf life of the beverage in the absence of preservatives.

Pomegranate juice also provides a source of natural polyphenols. In one aspect, pomegranate juice or extract is a natural source of polyphenols, including polyphenol antioxidants. Polyphenols from pomegranate juice and extracts include the class of molecules called ellagitannins, which have health benefits including reductions in low density lipoprotein (LDL) levels. In a further aspect, antioxidants and polyphenols from pomegranate juice have been linked to improvement in erectile dysfunction symptoms.

Pomegranate juice also includes B vitamins “B vitamins” include eight compounds—thiamine, riboflavin, niacin, pantothenic acid, pyridoxine, biotin, folate (or folic acid), and cobalamin—that are involved in cellular metabolism. B vitamin supplementation is thought to increase as part of a well-balanced diet low in saturated fat and cholesterol, it is thought that B vitamins including pyridoxine (B6), folate (B9), and cobalamin (B12) may reduce the risk of cardiovascular disease. Certain B complex vitamins, including but not limited to folate, are thought to provide a source of complementary energy for sexual performance and/or to improve sexual performance over time. In one aspect, the beverages disclosed herein include one or more B vitamins In a further aspect, the B vitamins included in the beverage come from the pomegranate juice or extract that is part of the beverage. In another aspect, one or more exogenously-sourced B vitamins (i.e., vitamins in addition to those present in pomegranate juice) can be added to the beverage to improve the nutrient profile.

In one aspect, one serving of a beverage described herein contains about eight fluid ounces of pomegranate juice. In yet another aspect, additional vitamins in the beverage are provided by the inclusion of pomegranate juice such as, for example, 33% of the USDA recommended daily allowance (RDA) of vitamin E, 13% of the RDA of folate, 30% of the RDA of vitamin B5, 20% of the RDA of vitamin B6, 9% of the RDA of vitamin A (as beta carotene), 30% of the RDA of vitamin C, and 10% of the RDA of vitamin K. In yet another aspect, additional minerals in the beverage are provided by the inclusion of pomegranate juice such as, for example, 1.2 mg of iron, 13% of the RDA of potassium, and 19% of the RDA of copper.

Although pomegranate juice imparts a satisfactory taste or flavor to the beverages described herein, one or more natural flavors and/or sweeteners can be added to the beverage.

The beverages described herein include ginseng. Ginseng, also known as “Asian ginseng,” “Korean ginseng,” and/or “Chinese ginseng,” has the scientific name Panax ginseng and has long been used in herbal medicine preparations. In one aspect, the ginseng useful herein is a dry powder derived entirely from ginseng root. For example, the ginseng root can be processed by as series of steaming and drying steps followed by grinding the resulting dried root into a powder.

In one aspect, the beverages disclosed herein contain ginseng powder (90%, 95%, 99%, or 100% purity) in the amount of 200 mg to 300 mg per eight fluid ounces of pomegranate juice. In another aspect, the ginseng powder can be present in the beverage in the amount of 200 mg, 225 mg, 250 mg, 275 mg, or 300 mg per eight fluid ounces of pomegranate juice, where any value can form a lower and upper-endpoint of a range (e.g., 220 mg to 275 mg, 225 mg to 275 mg, etc.).

The beverages described also include an extract of Maca root (Lepidium meyenii), which is also referred to as “Peruvian ginseng.” Maca root is a Peruvian plant related to radishes and turnips. In one aspect, the beverages include an extract of maca root in the amount of 400 mg to 600 mg per eight fluid ounces of pomegranate juice, wherein the extract of maca root is a dry powder standardized to at least 0.6% by weight glucosinates. In another aspect, the extract of maca root is a dry powder standardized from 0.6% to 2% by weight glucosinates.

In another aspect, the maca root extract can be present in the beverage in the amount of 400 mg, 450 mg, 500 mg, 550 mg, or 600 mg per eight fluid ounces of pomegranate juice, where any value can form a lower and upper-endpoint of a range (e.g., 450 mg to 550 mg, etc.). In a further aspect, the extract of maca root is a dry powder standardized to at least 0.6% by weight glucosinates present in the beverage in the amount of 400 mg, 450 mg, 500 mg, 550 mg, or 600 mg per eight fluid ounces of pomegranate juice, where any value can form a lower and upper-endpoint of a range (e.g., 450 mg to 550 mg, etc.).

The beverages described herein contain L-arginine or a pharmaceutically acceptable salt thereof. In one aspect, the L-arginine or pharmaceutically acceptable salt thereof is selected from L-arginine ethyl ester, L-arginine ethyl ester HCl, arginine HCl, L-arginine malate, L-arginine orotate, L-arginine hexanoate, L-arginine ketoisocaproic acid, L-arginine pyroglutamate, L-arginine taurinate, or a combination thereof.

In a further aspect, the L-arginine or pharmaceutically acceptable salt thereof (90%, 95%, 99%, or 100% purity) is present in an amount of from 300 mg to 500 mg per eight fluid ounces of pomegranate juice. In another aspect, L-arginine can be present in the beverage in the amount of 300 mg, 350 mg, 400 mg, 450 mg, or 500 mg per eight fluid ounces of pomegranate juice, where any value can form a lower and upper-endpoint of a range (e.g., 430 mg to 450 mg, etc.).

The beverages described herein can include one or more natural additives. In one aspect, the beverage can include Tongkat Ali. Tongkat Ali, which is Eurycoma longifolia, is also known as “Long Jack” or “Malaysian ginseng.” In one aspect, Tongkat Ali (90%, 95%, 99%, or 100% purity) is present in the beverages in the amount of 50 mg to 200 mg per eight fluid ounces of beverage. In another aspect, Tongkat Ali can be present in the beverage in the amount of 50 mg, 75 mg, 100 mg, 125 mg, 150 mg, 175 mg, or 200 mg per eight fluid ounces of pomegranate juice, where any value can form a lower and upper-endpoint of a range (e.g., 50 mg to 150 mg, etc.).

In another aspect, beverages described herein can include damiana leaf. Damiana leaf comes from a genus of shrubs, including species Turnera diffusa, T. aphrodisiaca, and T. microphylla, found in Mexico, the southern United States, and in some parts of South America. In one aspect, the beverages disclosed herein can include damiana leaf (90%, 95%, 99%, or 100% purity) in the amount of 200 mg to 400 mg per eight fluid ounces. In another aspect, damiana leaf can be present in the beverage in the amount of 200 mg, 225 mg, 250 mg, 275 mg, 300 mg, 325 mg, 350 mg, 375 mg, or 400 mg per eight fluid ounces of pomegranate juice, where any value can form a lower and upper-endpoint of a range (e.g., 250 mg to 350 mg, etc.).

The beverages described herein can be formulated for consumption by men and women. In one aspect, beverages for consumption by men include pomegranate juice, ginseng, maca root, L-arginine or a pharmaceutically acceptable salt thereof, and Tongkat Ali. In another aspect, the male beverage includes

(a) pomegranate juice;
(b) ginseng in the amount of 200 mg to 300 mg per eight fluid ounces of pomegranate juice;
(c) maca root in the amount of 400 mg to 600 mg per eight fluid ounces of pomegranate juice;
(d) L-arginine or a pharmaceutically acceptable salt thereof in the amount of 300 mg to 500 mg per eight fluid ounces of pomegranate juice; and
(e) tongkat ali in the amount of 50 mg to 200 mg per eight fluid ounces of pomegranate juice.

In another aspect, beverages for consumption by women include pomegranate juice, ginseng, maca root, L-arginine or a pharmaceutically acceptable salt thereof, Tongkat Ali, and damiana leaf. In another aspect, the female beverage includes

(a) pomegranate juice;
(b) ginseng in the amount of 200 mg to 300 mg per eight fluid ounces of pomegranate juice;
(c) maca root in the amount of 400 mg to 600 mg per eight fluid ounces of pomegranate juice;
(d) L-arginine or a pharmaceutically acceptable salt thereof in the amount of 300 mg to 500 mg per eight fluid ounces of pomegranate juice;
(e) tongkat ali in the amount of 50 mg to 200 mg per eight fluid ounces of pomegranate juice; an
(f) damiana leaf in the amount of 200 mg to 400 mg per eight fluid ounces of pomegranate juice.

In some aspects, the beverages disclosed herein include additional ingredients known in the art. In other aspects, the beverages are 100% natural (i.e., made with 100% natural components) with no artificial colors, dyes, preservatives, sweeteners, or the like.

A “sweetener” is a compound, extract, or composition that imparts a sweet taste to a food of beverage. Inclusion of a sweetener may assist in improving the flavor of a food or beverage product. In one aspect, the beverages disclosed herein do not contain any added sweeteners. In another aspect, the beverages disclosed herein contain one or more added sweeteners.

In one aspect, the sweetener is a natural sweetener. Examples of sweeteners useful herein include, but are not limited to, cane sugar, beet sugar, agave nectar, honey, maple syrup, molasses, barley malt syrup, brown rice syrup, date sugar, xylitol, erythritol, date sugar, lucuma powder, monk fruit extract, stevia extract, or any combination thereof. In one aspect, the sweetener can add calories or other nutrients such as, for example, fiber to the beverages. In an alternative aspect, the sweetener is a non-caloric sweetener.

A “preservative” is a compound or composition that is added to food and/or beverage products to prevent spoilage and prolong shelf life. In one aspect, a preservative can be an artificial or added preservative, such as, for example, sodium benzoate, benzoic acid, a nitrite, a sulfite, sodium sorbate, potassium sorbate, or a similar compound. In one aspect, added preservatives are not found in natural products. In another aspect, the beverages described herein do not contain any added or artificial preservatives.

“Natural flavors” are defined by the Code of Federal Regulations as essential oils, oleoresins, essences or extractives, protein hydrolysates, distillates, or any products of roasting, heating, or enzymolysis, which contain the flavoring constituents derived from a spice, fruit or fruit juice, vegetable or vegetable juice, edible yeast, herb, bark, bud, root, leaf, or similar plant material, meat, seafood, poultry, eggs, dairy products, or fermentation products thereof, whose significant function in food is flavoring rather than nutritional. In one aspect, the beverages disclosed herein include one or more added natural flavors. In an alternative aspect, the beverages disclosed herein are free from any added natural flavors other than those already possessed by the ingredients described herein.

In another aspect, additional components such as prohormones, fungi and/or fungal extracts, lichens and/or lichen extracts, and like compounds can be included in the beverages described herein. In an alternative aspect, the beverages do not contain fungi, fungal extracts, lichens, lichen extracts, or prohormones.

The beverages described herein can be made and bottled using techniques known in the art for making beverages for human consumption. In one aspect, the beverage is prepared by a hot-fill process. The hot-fill process is the procedure by which containers are filled with a beverage at a high temperature and capped soon thereafter to ensure continued sterility of the container and product during and after the fill process. Thus, no preservatives are needed or added to the beverage. In one aspect, the different components of the beverage (e.g., ginseng, L-arginine, maca root, etc.) are added to the pomegranate extract or juice under hot-fill conditions (e.g., about 89 ° C.). The hot composition is then introduced into a suitable container composed of, for example, a polyolefin (e.g., polyethylene, polypropylene), a polyester (e.g., PET), or glass. The hot-fill techniques disclosed in U.S. Pat. Nos. 7,000,793 and 6,339,952, which are incorporated by reference for their teachings, can be used herein. After the beverages have been introduced into the bottles and capped, the beverages can be stored at room temperature for several months.

The beverages described herein possess numerous health benefits. In one aspect, the beverages provided herein can enhance the sexual performance of a subject by consuming one or more servings of the beverages described herein. In one aspect, the beverages described herein can be consumed at least 48 hours prior to sexual activity in order to achieve enhanced sexual performance. In another aspect, the beverage can be consumed daily, every other day, or every three or four days to maintain enhanced sexual performance and promote cardiovascular health. In another aspect, the subject can drink one eight ounce serving of the beverages described herein per day in order to achieve the benefits of the beverage.

“Sexual performance” is associated with sexual desire, arousal, endurance or stamina, energy, frequency of sexual activity, and response, including the quality or intensity of orgasm, in both men and women. Sexual performance for both sexes may further relate to increased fertility. In both men and women, enhanced sexual performance can be associated with an increase in blood flow to or sensitivity of the genital organs. In some aspects, enhanced sexual performance for men includes ease of obtaining and/or maintaining an erection or, alternatively, a decrease in instances or severity of erectile dysfunction, premature ejaculation, and similar conditions. In another aspect, enhanced sexual performance for men includes increased testosterone levels and/or increase in sperm count. In other aspects, enhanced sexual performance for women is associated with increased lubrication or immune system and other physiological changes that enhance the woman's chances of becoming pregnant.

Enhancement of sexual performance can be objectively or subjectively assessed using tools such as, for example, self-assessments or questionnaires provided to subjects before and after consuming the beverages disclosed herein. Enhanced sexual performance can also be assessed by measuring certain physical and physiological indicators such as testosterone level and sperm count in men or changes in immunoglobulin levels and other immune system changes in women.

In another aspect, the beverages described herein reduce the risk of cardiovascular disease in a subject by consuming one or more servings of the beverages described herein. “Cardiovascular disease” includes, but is not limited to, a heart condition or condition of the blood vessels such as, for example, diseased vessels including those that are narrowed or blocked, chest pain, a structural problem, or a blood clot. Common cardiovascular diseases include, for example, coronary artery disease, high blood pressure or hypertension, congestive heart failure, arrhythmia, high cholesterol or hypercholesterolemia, peripheral artery disease, and congenital conditions. If left untreated, cardiovascular diseases can lead to heart attack, stroke, pulmonary hypertension, aortic aneurysm, or even cardiac arrest.

In some aspects, consumption of the beverages disclosed herein is associated with a reduced risk of cardiovascular disease. “Reducing” the risk of cardiovascular disease refers to making an item or quantity smaller or lesser in amount, degree, or size. For example, if an individual has risk level X, where X is between 0 (no likelihood of cardiovascular disease) and 1 (100% likelihood of developing cardiovascular disease) for cardiovascular disease based on genetics and lifestyle factors, reducing the risk would lead to new, lower risk level Y, such that 0<Y<X. In some aspects, the risk of developing cardiovascular disease is decreased for a subject consuming the beverages disclosed herein compared to the same subject when not consuming the beverages.

In still another aspect, the beverages described herein can simultaneously reduce the risk of cardiovascular disease in a subject while also enhancing the subject's sexual performance by consuming one or more servings of the beverages described herein.

Ingredients in a sample preparation of the beverages described herein are listed in Table 1 below. In one aspect, beverages prepared with ingredients in the amounts described in Table 1 are expected to be especially useful for enhancing sexual performance in male subjects.

TABLE 1
First Example Beverage

| Component | Amount per 8 fl oz. of Pomegranate Juice |
|---|---|
| Pomegranate Juice | — |
| Ginseng | 200 mg-300 mg |
| Maca Root | 400 mg-600 mg |
| L-arginine (or salt thereof) | 300 mg-500 mg |
| Tongkat Ali | 50 mg-200 mg |

Ingredients in a second sample preparation of the beverages described herein are listed in Table 2 below. In one aspect, beverages prepared with ingredients in the amounts described in Table 2 are expected to be especially useful for enhancing sexual performance in female subjects.

TABLE 2
Second Example Beverage

| Component | Amount per 8 fl oz. of Pomegranate Juice |
|---|---|
| Pomegranate Juice | — |
| Ginseng | 200 mg-300 mg |
| Maca Root | 400 mg-600 mg |
| L-arginine (or salt thereof) | 300 mg-500 mg |
| Tongkat Ali | 50 mg-200 mg |
| Damiana Leaf | 200 mg-400 mg |

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions, and methods disclosed herein. It is intended that the specification and examples be considered as exemplary.

What is claimed:

1. A beverage comprising (a) pomegranate juice, (b) ginseng, (c) maca root, and (d) L-arginine or a pharmaceutically acceptable salt thereof.

2. The beverage of claim 1, wherein the beverage further comprises tongkat ali.

3. The beverage of claim 1, wherein the beverage further comprises tongkat ali and damiana leaf.

4. The beverage of claim 1, wherein the beverage comprises:
(f) pomegranate juice;
(g) ginseng in the amount of 200 mg to 300 mg per eight fluid ounces of pomegranate juice;
(h) maca root in the amount of 400 mg to 600 mg per eight fluid ounces of pomegranate juice;
(i) L-arginine or a pharmaceutically acceptable salt thereof in the amount of 300 mg to 500 mg per eight fluid ounces of pomegranate juice; and
(j) tongkat ali in the amount of 50 mg to 200 mg per eight fluid ounces of pomegranate juice.

5. The beverage of claim 1, wherein the beverage comprises:
(g) pomegranate juice;
(h) ginseng in the amount of 200 mg to 300 mg per eight fluid ounces of pomegranate juice;
(i) maca root in the amount of 400 mg to 600 mg per eight fluid ounces of pomegranate juice;
(j) L-arginine or a pharmaceutically acceptable salt thereof in the amount of 300 mg to 500 mg per eight fluid ounces of pomegranate juice;
(k) tongkat ali in the amount of 50 mg to 200 mg per eight fluid ounces of pomegranate juice; and
(l) damiana leaf in the amount of 200 mg to 400 mg per eight fluid ounces of pomegranate juice.

6. The beverage of claim 1, wherein the beverage is:
(a) pomegranate juice;
(b) ginseng in the amount of 200 mg to 300 mg per eight fluid ounces of pomegranate juice;
(c) maca root in the amount of 400 mg to 600 mg per eight fluid ounces of pomegranate juice;
(d) L-arginine or a pharmaceutically acceptable salt thereof in the amount of 300 mg to 500 mg per eight fluid ounces of pomegranate juice; and
(e) tongkat ali in the amount of 50 mg to 200 mg per eight fluid ounces of pomegranate juice.

7. The beverage of claim 1, wherein the beverage is:
(a) pomegranate juice;
(b) ginseng in the amount of 200 mg to 300 mg per eight fluid ounces of pomegranate juice;
(c) maca root in the amount of 400 mg to 600 mg per eight fluid ounces of pomegranate juice;
(d) L-arginine or a pharmaceutically acceptable salt thereof in the amount of 300 mg to 500 mg per eight fluid ounces of pomegranate juice;
(e) tongkat ali in the amount of 50 mg to 200 mg per eight fluid ounces of pomegranate juice; and
(f) damiana leaf in the amount of 200 mg to 400 mg per eight fluid ounces of pomegranate juice.

8. The beverage of claim 1, wherein the beverage does not contain a preservative.

9. The beverage of claim 1, wherein the beverage the beverage is produced by a hot-fill process.

10. A method for reducing the risk of a cardiovascular disease in a subject comprising consuming the beverage of claim 1.

11. A method for enhancing the sexual performance of a subject comprising consuming the beverage of claim 1.

12. A method for reducing the risk of a cardiovascular disease in a subject and enhancing the sexual performance of the subject comprising consuming the beverage of claim 1.
